(19) United States
(12) Patent Application Publication
(10) Pub. No.: US 2026/0138963 A1
(43) Pub. Date: May 21, 2026

(54) COMPOSITIONS FOR LOW-TEMPERATURE DISTILLATION OF HIGH-BOILING-POINT MOLECULES

(71) Applicant: Natural Extraction Systems, LLC, Boulder, CO (US)

(72) Inventor: C. Russell Thomas, Boulder, CO (US)

(21) Appl. No.: 19/369,626

(22) Filed: Oct. 27, 2025

Related U.S. Application Data
(63) Continuation-in-part of application No. 18/553,182, now Pat. No. 12,453,931.
(63) Continuation of application No. 19/117,555.
(60) Provisional application No. 63/170,308, filed on Apr. 2, 2021.
(60) Provisional application No. 63/173,196, filed on Apr. 9, 2021.
(60) Provisional application No. 63/250,863, filed on Sep. 30, 2021.
(60) Provisional application No. 63/250,917, filed on Sep. 30, 2021.
(60) Provisional application No. 63/412,428, filed on Oct. 1, 2022.
(60) Provisional application No. 63/412,429, filed on Oct. 1, 2022.

(51) Int. Cl. C07D 311/10

(52) U.S. Cl. CPC C07D 311/10

(57) ABSTRACT

Various aspects of this disclosure relate to a composition, comprising a gas phase and a condensed phase, wherein the gas phase comprises a molecule; the condensed phase comprises the molecule; the gas phase has a temperature and a pressure; the molecule has a boiling point at the pressure of the gas phase; the boiling point of the molecule is less than the temperature of the gas phase; the molecule has a vapor pressure at the temperature of the gas phase; the vapor pressure of the molecule is less than the pressure of the gas phase; the condensed phase consists of one or both of a solid phase and a liquid phase; the condensed phase is suspended in the gas phase; and the condensed phase has a surface-area-to-volume ratio of at least 500 per meter.

### CROSS-REFERENCE TO RELATED APPLICATIONS

This patent application is a continuation of U.S. patent application Ser. No. 19/117,555, filed Apr. 1, 2025, which is the U.S. National Phase under 35 U.S.C. §371 of International Application No. PCT/US2023/034319, which claims priority to U.S. Provisional Patent Application No. 63/412,428 , filed Oct. 1, 2022, and U.S. Provisional Patent Application No. 63/412,429 , filed Oct. 1, 2022, each of which is incorporated by reference in its entirety.

This patent application also claims priority to U.S. patent application Ser. No. 18/553,182, which granted as U.S. Pat. No. 12,453,931, and which is the U.S. National Phase under 35 U.S.C. § 371 of International Application No. PCT/US2022/023140, which claims priority to U.S. Provisional Patent Application No. 63/170,308, filed Apr. 2, 2021; U.S. Provisional Patent Application No. 63/173,196, filed Apr. 9, 2021; U.S. Provisional Patent Application No. 63/250,863, filed Sep. 30, 2021; and U.S. Provisional Patent Application No. 63/250,917, filed Sep. 30, 2021, each of which is incorporated by reference in its entirety.

### BACKGROUND

Natural product extraction is a mature technical field. The last major innovation was the development of supercritical fluid extraction in the 1980's. An oligopoly of a few major flavor and fragrance companies achieved economies of scale and global footprints. Their products became commoditized and fungible, which forced cost-cutting and tight profit margins.

Many “current” good manufacturing practices are becoming anachronistic as the global supply chain increasingly demands sustainable agriculture, fair trade, and organic certification. Regulators and consumers increasingly pressure the ingredients industries, and newsworthy publications suggest that new regulations may inadvertently end the economically-viable production of several key ingredients used in everyday consumer packaged goods.

A new extraction technology that could increase profit margins without reliance upon disfavored manufacturing practices could allow flavor and fragrance companies to absorb the cost of additional regulation and save the global supply of ingredients that currently require manufacturing methods that may not remain tenable in the years to come.

### SUMMARY

Various aspects of this disclosure relate to the discovery that converting a solid or a liquid into an aerosol allows the distillation of molecules from the aerosol in seconds at temperatures that are significantly less than the boiling points of the molecules. This discovery extends beyond aerosols to many compositions that have large surface-area-to-volume ratios.

### DETAILED DESCRIPTION

Various aspects of this disclosure relate to a composition, comprising a gas phase and a condensed phase, wherein: the gas phase comprises a molecule; the condensed phase comprises the molecule; the gas phase has a temperature and a pressure; the molecule has a boiling point at the pressure of the gas phase; the boiling point of the molecule is greater than the temperature of the gas phase; the molecule has a vapor pressure at the temperature of the gas phase; the vapor pressure of the molecule is less than the pressure of the gas phase; the condensed phase consists of one or both of a solid phase and a liquid phase; the condensed phase is suspended in the gas phase; and the condensed phase has a surface-area-to-volume ratio of at least 500 per meter. Such compositions may be prepared, for example, in an extraction chamber of an extraction machine described in European U.S. Pat. No. 3,283,606B1 , which is incorporated by reference in its entirety. This disclosure shall not be limited, however, to compositions that may be made in extraction machines according to European U.S. Pat. No. 3,283,606B 1 .

In this disclosure, the term “boiling point” includes both the conventional definition of the term, and, when a molecule of this disclosure lacks a boiling point and has a sublimation point, then the term “boiling point” encompasses the term sublimation point in reference to such a molecule. Caffeine sublimes instead of boiling, for example, and, in this disclosure, the “boiling point” of caffeine refers to the sublimation point of caffeine.

In some embodiments, solid or liquid particles of a condensed phase are introduced into a chamber, passageway, vessel, or tube that contains a moving gas phase. Bombardment of the condensed phase with the gas phase aerosolizes the condensed phase in the gas phase to produce a composition according to this disclosure and transports the composition through the chamber, passageway, vessel, or tube. Molecules evaporate from the condensed phase to form a vapor in the gas phase. A method is provided to separate the gas phase from the condensed phase, for example, such as by passing the composition through a cyclone. A method is then provided to separate the vapor from the rest of the gas phase. The gas phase containing the vaporized molecules may be passed through a spray or curtain of collection solvent or bubbled through a tank of collection solvent to condense the vaporized molecules into condensed molecules. The collection solvent may contain compounds that attract and absorb the vaporized molecules, thereby capturing a substantial portion of the vaporized molecules from the gas phase and holding the condensed molecules in the collection solvent. A collection solvent may comprise ethanol, a mixture of ethanol and water, or other solvents or mixtures of solvents. The collection solvent may be advantageously selected from solvents or a mixture of solvents that the evaporated molecules are dissolvable or miscible within, for example, to facilitate pumping of the condensed molecules in the collection solvent, but such features do not limit this disclosure. Collection solvents are further described in European U.S. Pat. No. 3,283,606B1 , which is incorporated its entirety by reference. Over time, more and more evaporated molecules are transferred from the gas phase to the collection solvent. Once the collection solvent has captured a sufficient quantity of the vaporized molecules, the collection solvent containing the condensed molecules may be removed from the extraction machine, either manually or automatically, and fresh collection solvent may be introduced back into the machine either manually or automatically. The removal and introduction of collection solvent may be performed in incremental batches or continuously.

“Comprising” refers to an open set, for example, such that a gas phase that comprises a molecule can also comprise a second molecule.

“Consists” refers to a closed set, for example, such that a condensed phase that consists of one or both of a solid phase and a liquid phase cannot also comprise a plasma phase.

“Condensed phase” refers to “one or both of a solid phase and a liquid phase” without limitation, and “condensed phase” shall not be interpreted as implying that any or all of the condensed phase originated from a gas phase that condensed except as explicitly set forth in this disclosure.

In some embodiments, the composition has a vaporization rate for conversion of the molecule in the condensed phase into the molecule in the gas phase; the composition has a condensation rate for conversion of the molecule in the gas phase into the molecule in the condensed phase; the composition has a mass transfer rate, which is equal to the vaporization rate minus the condensation rate; and the mass transfer rate is a positive number.

In some embodiments, the mass transfer rate is at least 5 micrograms of the molecule per gram of the composition per second. In some embodiments, the mass transfer rate is at least 5 micrograms of the molecule per gram of the condensed phase per second. In some embodiments, the mass transfer rate is at least 5 micrograms of the molecule per gram of the gas phase per second.

In some embodiments, the temperature of the gas phase is at least 25 degrees Celsius.

In some embodiments, the temperature of the gas phase is no greater than 250 degrees Celsius.

In some embodiments, the temperature of the gas phase is greater than 100 and no greater than 250 degrees Celsius. In some specific embodiments, the temperature of the gas phase is greater than 100 and no greater than 235 degrees Celsius.

In some embodiments, the condensed phase has a temperature that is less than the temperature of the gas phase.

In some embodiments, the composition has a nonzero rate of sensible heat transfer from the gas phase to the condensed phase.

In some embodiments, the composition has a rate of sensible heat transfer from the gas phase to the condensed phase of at least 2 joules per gram of the condensed phase per second. In some specific embodiments, the composition has a rate of sensible heat transfer from the gas phase to the condensed phase of at least 20 joules per gram of the condensed phase per second.

In some embodiments, the composition has a rate of sensible heat transfer from the gas phase to the condensed phase of no greater than 20 kilojoules per gram of the condensed phase per second. In some specific embodiments, the composition has a rate of sensible heat transfer from the gas phase to the condensed phase of no greater than 2 kilojoules per gram of the condensed phase per second. In some specific embodiments, the composition has a rate of sensible heat transfer from the gas phase to the condensed phase of no greater than 1 kilojoule per gram of the condensed phase per second. In some specific embodiments, the composition has a rate of sensible heat transfer from the gas phase to the condensed phase of no greater than 500 joules per gram of the condensed phase per second. In some specific embodiments, the composition has a rate of sensible heat transfer from the gas phase to the condensed phase of no greater than 100 joules per gram of the condensed phase per second. In some specific embodiments, the composition has a rate of sensible heat transfer from the gas phase to the condensed phase of no greater than 50 joules per gram of the condensed phase per second. In some specific embodiments, the composition has a rate of sensible heat transfer from the gas phase to the condensed phase of no greater than 20 joules per gram of the condensed phase per second.

In some embodiments, the composition has a nonzero rate of latent heat transfer between the gas phase and the condensed phase. In some specific embodiments, the composition has a rate of latent heat transfer between the gas phase and the condensed phase of at least 10 millijoules per gram of the condensed phase per second. In some very specific embodiments, the composition has a rate of latent heat transfer between the gas phase and the condensed phase of at least 100 millijoules per gram of the condensed phase per second. In some specific embodiments, the composition has a rate of latent heat transfer between the gas phase and the condensed phase of no greater than 2 kilojoules per gram of the condensed phase per second. In some specific embodiments, the composition has a rate of latent heat transfer between the gas phase and the condensed phase of no greater than 1 kilojoule per gram of the condensed phase per second. In some very specific embodiments, the composition has a rate of latent heat transfer between the gas phase and the condensed phase of no greater than 500 joules per gram of the condensed phase per second. In some very specific embodiments, the composition has a rate of latent heat transfer between the gas phase and the condensed phase of no greater than 200 joules per gram of the condensed phase per second.

In some embodiments, the condensed phase is introduced to a chamber, tube, or passageway that contains a moving gas phase. Upon entering the chamber, tube, or passageway, the condensed phase is bombarded by the gas phase. The bombardment by the gas phase aerosolizes the condensed phase in the gas phase. A portion of the condensed phase is translated and carried by the gas phase through the chamber, tube, or passageway. To facilitate rapid evaporation of a molecule from the condensed phase into a vapor, the gas phase may be heated. Additional heat may be optionally applied to surfaces of the chamber, tube, or passageway to apply additional energy to the composition. The heated gas phase contacts the condensed phase for a period of time and has an average temperature during the time of contact. The total combined heating energy applied by the system to heat both the gas phase and the condensed phase may be as follows: when the average temperature is less than 275 degrees Celsius, then the combined energy applied to the gas phase and the condensed phase is less than 100 kilojoules per gram of the condensed phase; when the average temperature is less than 251 degrees Celsius, then the energy applied to the gas phase and the condensed phase is less than 80 kilojoules per gram of the condensed phase; when the average temperature is less than 225 degrees Celsius, then the energy applied to the gas phase and the condensed phase is less than 70 kilojoules per gram of the condensed phase; when the average temperature is less than 200 degrees Celsius, then the energy applied to the gas phase and the condensed phase is less than 65 kilojoules per gram of the condensed phase; when the average temperature is less than 175 degrees Celsius, then the energy applied to the gas phase and the condensed phase is less than 55 kilojoules per gram of the condensed phase; when the average temperature is less than 150 degrees Celsius, then the energy applied to the gas phase and the condensed phase is less than 50 kilojoules per gram of the condensed phase; when the average temperature is less than 125 degrees Celsius, then the energy applied to the gas phase and the condensed phase is less than 45 kilojoules per gram of the condensed phase; when the average temperature is less than 100 degrees Celsius, then the energy applied to the gas phase and the condensed phase is less than 35 kilojoules per gram of the condensed phase; when the average temperature is less than 75 degrees Celsius, then the energy applied to the gas phase and the condensed phase is less than 30 kilojoules per gram of the condensed phase; when the average temperature is less than 50 degrees Celsius, then the energy applied to the gas phase and the condensed phase is less than 20 kilojoules per gram of the condensed phase; and when the average temperature is less than 35 degrees Celsius, then the energy applied to the gas phase and the condensed phase is less than 10 kilojoules per gram of the condensed phase. In some embodiments, the gas phase has a pressure of at least 0.1 and no greater than 100 atmospheres. In some specific embodiments, the gas phase has a pressure of at least 0.5 and no greater than 2 atmospheres. In some very specific embodiments, the gas phase has a pressure of at least 0.75 and no greater than 1.25 atmospheres.

In some embodiments, the composition has an altitude; the altitude has an atmospheric pressure; and the pressure of the gas phase is greater than the atmospheric pressure at the altitude.

In some embodiments, the condensed phase has a surface-area-to-volume ratio of at least 1000 per meter. In some specific embodiments, the condensed phase has a surface-area-to-volume ratio of at least 2400 per meter. In some even more specific embodiments, the condensed phase has a surface-area-to-volume ratio of at least 5000 per meter. In some very specific embodiments, the condensed phase has a surface-area-to-volume ratio of at least 10,000 per meter.

In some embodiments, the condensed phase has an average particle size of no greater than 5 millimeters. In some specific embodiments, the condensed phase has an average particle size of no greater than 500 micrometers.

“Particle size” refers to the longest linear distance that connects one point of a particle of the composition to another point of the particle in three-dimensional Euclidean space.

In some embodiments, the condensed phase has an average terminal velocity of no greater than 5 meters per second in still, dry air at 1 atmosphere of pressure. In some specific embodiments, the condensed phase has an average terminal velocity of no greater than 1 meter per second in still, dry air at 1 atmosphere of pressure.

In some embodiments, the composition has a turbulent flow.

In some embodiments, the composition has an average Reynolds number that is greater than 100. In some specific embodiments, the composition has an average Reynolds number that is greater than 1000. In some very specific embodiments, the composition has an average Reynolds number that is greater than 10,000.

In some embodiments, the composition has an average Reynolds number that is no greater than 1,000,000.

In some embodiments, the composition has an average drag coefficient of at least 0.5.

In some embodiments, the composition has a laminar flow.

In some embodiments, the composition has an average velocity of at least 100 millimeters per second. In some specific embodiments, the composition has an average velocity of at least 1 meter per second.

In some embodiments, the composition has an average kinetic energy of at least 5 microjoules per gram of the composition. In some specific embodiments, the composition has an average kinetic energy of at least 500 microjoules per gram of the composition.

In some embodiments, the composition has an average kinetic energy of no greater than 50 joules per gram of the composition. In some specific embodiments, the composition has an average kinetic energy of no greater than 5 joules per gram of the composition.

In some embodiments, the composition comprises at least 1 gram of the condensed phase per cubic meter of the gas phase.

In some embodiments, the composition comprises no greater than 1 gram of the condensed phase per liter of the gas phase.

In some embodiments, the composition comprises at least 2 micrograms of the molecule per gram of the condensed phase.

In some embodiments, the composition comprises no greater than 200 milligrams of the molecule per gram of the condensed phase.

In some embodiments, the composition comprises at least 2 micrograms of the molecule per gram of the gas phase.

In some embodiments, the composition comprises no greater than 200 milligrams of the molecule per gram of the gas phase.

In some embodiments, the gas phase comprises at least 10 percent of the molecule of the composition. In some specific embodiments, the gas phase comprises at least 25 percent of the molecule of the composition. In some very specific embodiments, the gas phase comprises at least 50 percent of the molecule of the composition.

In some embodiments, the gas phase comprises a greater percentage of the molecule than the condensed phase.

In some embodiments, the condensed phase comprises one or more polysaccharides, disaccharides, or monosaccharides. In some specific embodiments, the condensed phase comprises cellulose. In some very specific embodiments, the condensed phase comprises cellulose I.

In some embodiments, the condensed phase comprises dextrin.

In some embodiments, the condensed phase comprises pyrodextrin.

In some embodiments, the condensed phase comprises levoglucosan.

In some embodiments, the condensed phase comprises one or more proteins, peptides, or amino acids. In some specific embodiments, the condensed phase comprises one or more proteins that comprise amino acid sequences that encode a cellulose synthase.

In some embodiments, the condensed phase comprises one or more nucleic acids, nucleotides, or nucleobases. In some specific embodiments, the condensed phase comprises one or more nucleic acids that comprise nucleotide sequences that encode a cellulose synthase.

In some embodiments, the condensed phase comprises 1, 2, 3, 4, 5, or each of sodium ion (“Na+”), potassium ion (“K+”), calcium ion (“Ca++”), magnesium ion (“Mg++), monohydrogen phosphate, and dihydrogen phosphate. In some specific embodiments, the condensed phase comprises monohydrogen phosphate and dihydrogen phosphate.

In some embodiments, the condensed phase comprises chlorophyll.

In some embodiments, the condensed phase comprises cells. In some specific embodiments, the condensed phase comprises cells, and each cell comprises a cell wall. In some very specific embodiments, the condensed phase comprises cells, and the cells are plant cells.

In some embodiments, the condensed phase comprises cells; the cells comprise intact cells that each comprise an intracellular space and an intact cell membrane that inhibits fluid communication between the intracellular space and the gas phase; and the cells comprise lysed cells that each comprise an intracellular space and a lysed cell membrane that allows fluid communication between the intracellular space and the gas phase.

In some embodiments, at least 10 percent of the cells are lysed cells. In some specific embodiments, at least 25 percent of the cells are lysed cells. In some very specific embodiments, at least 50 percent of the cells are lysed cells.

In some embodiments, the intracellular space of each intact cell comprises a volatile molecule; the volatile molecule has a boiling point at the pressure of the gas phase; and the boiling point of the volatile molecule at the pressure of the gas phase is less than the temperature of the gas phase.

In some embodiments, the intracellular space of each intact cell comprises a superheated liquid.

In some embodiments, the intracellular space of each intact cell comprises a gas.

In some embodiments, the intracellular space of each intact cell comprises water; the water has a boiling point at the pressure of the gas phase; and the boiling point of water at the pressure of the gas phase is less than the temperature of the gas phase.

In some embodiments, the intracellular space of each intact cell comprises superheated water.

In some embodiments, the intracellular space of each intact cell comprises water vapor.

In some embodiments, the intracellular space of each intact cell has a pressure that is greater than the pressure of the gas phase.

In some embodiments, the composition has a nonzero cell lysis rate for conversion of intact cells into lysed cells. In some specific embodiments, the composition has a cell lysis rate for conversion of intact cells into lysed cells; and the cell lysis rate is at least 1 percent of the cells per second. In some very specific embodiments, the composition has a cell lysis rate for conversion of intact cells into lysed cells; and the cell lysis rate is at least 10 percent of the cells per second. Cell lysis improves extraction efficiency by facilitating fluid communication between the intracellular space and the gas phase.

In some embodiments, the gas phase comprises one or more of molecular nitrogen, molecular oxygen, carbon dioxide, argon, neon, water vapor, and ethanol vapor. In some specific embodiments, the gas phase comprises one or more of the molecule, molecular nitrogen, molecular oxygen, carbon dioxide, argon, neon, water vapor, and ethanol vapor at a combined concentration of at least 50 percent by mass.

In some embodiments, the gas phase comprises molecular nitrogen. In some specific embodiments, the gas phase comprises the molecule and molecular nitrogen at a combined concentration of at least 50 percent by mass.

In some embodiments, the gas phase lacks water vapor at a concentration greater than 90 percent by mass. In some specific embodiments, the gas phase lacks water vapor at a concentration greater than 50 percent by mass. In some very specific embodiments, the gas phase lacks water vapor at a concentration greater than 10 percent by mass.

In some embodiments, the gas phase comprises dry steam. In some specific embodiments, the gas phase comprises the molecule and dry steam at a combined concentration of at least 50 percent by mass.

In some embodiments, the molecule is a furan.

In some embodiments, the molecule is furyl-hydroxymethyl ketone (CAS: 17678-19-2); 2-methyl-benzofuran (CAS: 4265-25-2); 2-(2-furanylmethyl)-5-methyl-furan (CAS 13678-51-8); or 2,5-furandicarboxaldehyde (CAS: 823-82-5).

In some embodiments, the molecule is an alcohol.

In some embodiments, the molecule is nonan-1-ol (CAS: 143-08-8); decan-1-ol (CAS: 112-53-8); dodec-1-ol (CAS: 112-53-8); tetradecane-1-ol (CAS: 112-72-1); hexadecane-1-ol (CAS: 36653-82-4); or octadecane-1-ol (CAS: 112-92-5).

In some embodiments, the molecule is a fatty alcohol that comprises at least 9 carbon atoms.

In some embodiments, the molecule is nonen-3-ol (CAS: 21964-44-3).

In some embodiments, the molecule is an unsaturated alcohol.

In some embodiments, the molecule is 2-decen-1-ol (CAS: 22104-80-9).

In some embodiments, the molecule is an aromatic alcohol.

In some embodiments, the molecule is an aldehyde.

In some embodiments, the molecule is an unsaturated aldehyde.

In some embodiments, the molecule is a substituted aldehyde.

In some embodiments, the molecule is a unsubstituted aldehyde.

In some embodiments, the molecule is tetradecanal (CAS: 124-25-4); (Z)-2-decenal (CAS: 2497-25-8); or (E, E)-2,4-decadienal (CAS: 25152-84-5).

In some embodiments, the molecule is a carboxylic acid.

In some embodiments, the molecule is a low molecular weight volatile acid.

In some embodiments, the molecule is a fatty acid.

In some embodiments, the molecule is an unsaturated fatty acid.

In some embodiments, the molecule is a saturated fatty acid.

In some embodiments, the molecule is octanoic acid (CAS: 124-07-2); nonanoic acid (CAS: 112-05-0); n-decanoic acid (CAS: 334-48-5); n-hexadecanoic acid (CAS: 57-10-3); heptadecanoic acid (CAS: 506-12-7); or octadecanoic acid (CAS: 57-11-4).

In some embodiments, the molecule is a fatty acid.

In some embodiments, the molecule is a substituted carboxylic acid.

In some embodiments, the molecule is 2-ethylhexanoic acid (CAS: 149-57-5).

In some embodiments, the molecule is an unsaturated carboxylic acid.

In some embodiments, the molecule is trans-2-undecenoic acid (CAS: 15790-94-0).

In some embodiments, the molecule is an aromatic acid.

In some embodiments, the molecule is benzoic acid (CAS: 65-85-0); or phthalic acid (CAS: 88-99-3).

In some embodiments, the molecule is an ester.

In some embodiments, the molecule is a branched ester.

In some embodiments, the molecule is a non-branched ester.

In some embodiments, the molecule is a primary, secondary, tertiary, substituted, or unsubstituted, ester.

In some embodiments, the molecule is a mono-ester.

In some embodiments, the molecule is a di-ester.

In some embodiments, the molecule is an ester of a saturated acid.

In some embodiments, the molecule is an ester of an unsaturated acid.

In some embodiments, the molecule is an ester of an aromatic acid.

In some embodiments, the molecule is methyl nonanoate (CAS: 1731-84-6); octyl butanoate (CAS: 110-39-4); isopropyl myristate (CAS: 110-27-0); ethyl nicotinate (CAS: 614-18-6); 3-hexenyl butanoate (CAS: 53398-84-8); diethyl butanedioate (CAS: 123-25-1); or diethyl itaconate (CAS: 2409-52-1).

In some embodiments, the molecule is a chemical derived from lignin.

In some embodiments, the molecule is a substituted phenol.

In some embodiments, the molecule is methyl salicylate (CAS: 119-36-8); eugenol (CAS 97-53-0); vanillin (CAS 121-33-5); (Z)-isoeugenol (CAS: 5932-68-3); ethyl anisate (CAS: 94-30-4); 4-methylguauacol (CAS: 93-51-6); or (E)-2,6-dimethoxy-4-(prop-1-en-1-yl)phenol (CAS: 20675-95-0).

In some embodiments, the molecule is a terpene.

In some embodiments, the molecule is 1,3,8-p-menthatriene, (CAS: 18368-95-1).

In some embodiments, the molecule is a monoterpene.

In some embodiments, the molecule is a sesquiterpene.

In some embodiments, the molecule is an oxide of a terpene.

In some embodiments, the molecule is an alkaloid.

In some embodiments, the molecule is a nitrogen-containing volatile compound.

In some embodiments, the molecule is caffeine (CAS: 58-08-2).

In some embodiments, the molecule is nicotine (CAS: 54-11-5).

In some embodiments, the molecule is 1H-pyrrole-2-carboxaldehyde (CAS: 1003-29-8).

In some embodiments, the molecule is a phytosterol.

In some embodiments, the molecule is a derivative of a phytosterol.

In some embodiments, the molecule is (4R,5R)-5-butyl-4-methyloxolan-2-one (CAS: 55013-32-6); (4S,5R)-5-butyl-4-methyloxolan-2-one (CAS: 39638-67-0); β-ionone (CAS: 8013-90-9); or δ-tetradecalactone (CAS: 2721-22-4).

In some embodiments, the molecule is an organosulfur compound.

In some embodiments, the molecule is bis(2-furfuryl)disulfide (CAS: 4437-20-1).

In some embodiments, the molecule is an alkane.

In some embodiments, the molecule is an alkene.

In some embodiments, the molecule is a hydrocarbon.

In some embodiments, the molecule is hydrocoumarin (CAS: 119-84-6).

In some embodiments, the molecule is coumarin (CAS: 91-64-5).

In some embodiments, the molecule is acetophenone (CAS: 98-86-2); alpha-bergamotol (CAS: 88034-74-6); alpha-bisabolol (CAS: 515-69-5); alpha-bisabolol oxide A (CAS: 22567-36-8); alpha-cadinol (CAS: 481-34-5); alpha-curcumene (CAS: 644-30-4); alpha-fenchene (CAS: 471-84-1); alpha-phellandrene (CAS: 99-83-2); alpha-pinene (CAS: 80-56-8); alpha-santalol (CAS: 115-71-9); alpha-terpinene (CAS: 99-86-5); alpha-terpineol (CAS: 98-55-5); alpha-terpinyl acetate (CAS: 80-26-2); alpha-thujene (CAS: 2867-05-2); alpha-thujone (CAS: 546-80-5); alpha-zingiberene (CAS: 495-60-3); azulene (CAS: 275-51-4); benzyl acetate (CAS: 140-11-4); benzyl benzoate (CAS: 120-51-4); bergamotene (CAS: 6895-56-3); beta-bisabolene (CAS: 495-61-4); beta-caryophyllene (CAS: 87-44-5); beta-damascenone (CAS: 23696-85-7); beta-eudesmol (CAS: 473-15-4); beta-farnesene (CAS: 77129-48-7); beta-phellandrene (CAS: 555-10-2); beta-pinene (CAS: 127-91-3); beta-santalol (CAS: 11031-45-1); beta-selinene (CAS: 17066-67-0); beta-sesquiphellandrene (CAS: 20307-83-9); beta-terpinene (CAS: 99-84-3); beta-terpinyl acetate (CAS: 10198-23-9); beta-thujene (CAS: 28634-89-1); beta-thujone (CAS: 1125-12-8); borneol (CAS: 464-45-9); bornyl acetate (CAS: 76-49-3); camphene (CAS: 79-92-5); camphor (CAS: 76-22-2); capsaicin (CAS: 404-86-4); carene (CAS: 13466-78-9); carvacrol (CAS: 499-75-2); carvone (CAS: 99-49-0); caryophyllene oxide (CAS: 1139-30-6); cedrene (CAS: 469-61-4); cedrol (CAS: 77-53-2); chamazulene (CAS: 529-05-5); chavicol (CAS: 501-92-8); cinnamaldehyde (CAS: 104-55-2); citral (CAS: 5392-40-5); citronellal (CAS: 106-23-0); citronellol (CAS: 106-22-9); citronellyl formate (CAS: 105-85-1); curzerene (CAS: 17910-09-7); cyclopentadecanolide (CAS: 106-02-5); decanal (CAS: 112-31-2); delta-guaiene (CAS: 3691-11-0); ethyl cinnamate (CAS: 103-36-6); eugenol (CAS: 97-53-0); farnesene (CAS: 502-61-4); farnesol (CAS: 4602-84-0); furanoeudesma-1,3-diene (CAS: 87605-93-4); furfural (CAS: 98-01-1); furfuryl acetate (CAS: 623-17-6); gamma-decalactone (CAS: 706-14-9); gamma-muurolene (CAS: 30021-74-0); gamma-nonalactone (CAS: 104-61-0); gamma-terpinene (CAS: 99-85-4); gamma-terpinyl acetate (CAS: 10235-63-9); geraniol (CAS: 106-24-1); geranyl acetate (CAS: 105-87-3); germacrene A (CAS: 28387-44-2); germacrene D (CAS: 37839-63-7); guaiacol (CAS: 90-05-1); heneicosane (CAS: 629-94-7); humulene (CAS: 6753-98-6); isoamyl benzoate (CAS: 94-46-2); kessane (CAS: 3321-66-2); limonene (CAS: 6876-12-6); linalool (CAS: 78-70-6); linalool oxide (CAS: 1365-19-1); linalyl acetate (CAS: 115-95-7); menthol (CAS: 89-78-1); menthone (CAS: 89-80-5); methyl cinnamate (CAS: 103-26-4); methyl eugenol (CAS: 93-15-2); methylpyrazine (CAS: 109-08-0); myrcene (CAS: 123-35-3); myristicin (CAS: 607-91-0); neral (CAS: 5392-40-5); nerol (CAS: 106-25-2); nerolidol (CAS: 7212-44-4); nookatone (CAS: 91416-23-8); nootkatin (CAS: 4431-03-2); nootkatol (CAS: 53643-07-5); nootkatone (CAS: 91416-23-8); ocimene (CAS: 7216-56-0); octanal (CAS: 124-13-0); para-cresol (CAS: 106-44-5); para-cymene (CAS: 99-87-6); patchouli alcohol (CAS: 5986-55-0); perillene (CAS: 539-52-6); phenylacetaldehyde (CAS: 122-78-1); phenylacetic acid (CAS: 103-82-2); phenylethyl alcohol (CAS: 60-12-8); phytol (CAS: 150-86-7); sabinene (CAS: 3387-41-5); safrole (CAS: 94-59-7); tau-muurolol (CAS: 19912-62-0); terpinen-4-ol (CAS: 562-74-3); terpinolene (CAS: 586-62-9); thymol (CAS: 89-83-8); valencene (CAS: 4630-07-3); vanillin (CAS: 121-33-5); zingerone (CAS: 122-48-5); zingiberenol (CAS: 58334-55-7); zingiberol (CAS: 6754-68-3); 1,8-cineole (CAS: 470-82-6); 1-phenylethyl acetate (CAS: 93-92-5); 2,6-dimethylpyrazine (CAS: 108-50-9); 2-furanmethanol (CAS: 98-00-0); 2-heptanol (CAS: 543-49-7); 2-heptanone (CAS: 110-43-0); 2-heptyl acetate (CAS: 5921-82-4); 2-methoxy-4-vinylphenol (CAS: 7786-61-0); 2-methyl-3-buten-2-ol (CAS: 115-18-4); 2-methylbutanoic acid (CAS: 116-53-0); 2-nonanone (CAS: 821-55-6); 2-pentanol (CAS: 6032-29-7); 2-pentyl acetate (CAS: 626-38-0); 2-phenylethyl alcohol (CAS: 60-12-8); 2-undecanone (CAS: 112-12-9); 3-methylbutanoic acid (CAS: 503-74-2); 3-phenylpropanoic acid (CAS: 501-52-0); 4-methylguaiacol (CAS: 93-51-6); 5-methylfurfural (CAS: 620-02-0); 6-gingerol (CAS: 23513-14-6); 6-methyl-5-hepten-2-one (CAS: 110-93-0); or 6-shogaol (CAS: 555-66-8).

In some embodiments, the molecule is phenylacetaldehyde oxime (CAS: 7028-48-0).

In some embodiments, the molecule is dihydrofarnesal (CAS: 32480-08-3).

In some embodiments, the molecule is not a cannabinoid carboxylic acid, tetrahydrocannabinolic acid, tetrahydrocannabivarin carboxylic acid, tetrahydrocannabiorcolic acid, cannabidiolic acid, cannabidivarin carboxylic acid, cannabidiorcolic acid, cannabichromenic acid, cannabichromevarinic acid, cannabigerolic acid, cannabigerovarinic acid, cannabicyclolic acid, cannabielsoic acid, perrottetinenic acid, cannabinolic acid, cannabivarin carboxylic acid, a carboxylate of any one of the preceding molecules, a cannabinoid, tetrahydrocannabinol, tetrahydrocannabivarin, tetrahydrocannabiorcol, cannabidiol, cannabidivarin, cannabidiorcol, cannabichromene, cannabichromevarin, cannabigerol, cannabigerovarin, cannabicyclol, cannabielsoin, perrottetinene, cannabinol, cannabivarin, a naturally-occurring ether of any one of the preceding molecules, a stereoisomer of any one of the preceding molecules; beta-caryophyllene, humulene, (−)-guaiol, (−)-alpha-bisabolol, linalool, alpha-terpineol, caryophyllene oxide, myrcene, eucalyptol, limonene, nerolidol, alpha-terpinene, borneol, (−)-isopulegol, delta-3-carene, para-cymene, or terpinolene.

In some embodiments, the composition lacks cannabis and any chemical species derived from cannabis at a concentration greater than 10 percent by mass. In some specific embodiments, the composition lacks cannabis and any chemical species derived from cannabis at a concentration greater than 1 percent by mass. In some very specific embodiments, the composition lacks cannabis and any chemical species derived from cannabis.

“Cannabis” refers to plants of the genus cannabis and any portion of a plant of the genus cannabis. Cannabis includes, for example, marijuana and industrial hemp.

“Any chemical species derived from cannabis” includes, for example chemical species that are extracted from cannabis and chemical species that are manufactured from cannabis such as by decarboxylating a cannabis extract comprising one or more cannabinoid carboxylic acids.

In some embodiments, the condensed phase comprises biomass of a perennial plant.

In some embodiments, the condensed phase comprises biomass of an annual plant.

In some embodiments, the condensed phase comprises agave.

In some embodiments, the condensed phase comprises mescal bagasse or tequila bagasse.

In some embodiments, the condensed phase comprises sugarcane.

In some embodiments, the condensed phase comprises sugarcane bagasse.

In some embodiments, the condensed phase comprises sorghum bagasse.

In some embodiments, the condensed phase comprises peat or smoked peat.

In some embodiments, the condensed phase comprises malted grain.

In some embodiments, the condensed phase comprises barley.

In some embodiments, the condensed phase comprises or malted barley.

In some embodiments, the condensed phase comprises corn.

In some embodiments, the condensed phase comprises corn fibers, corn cobs, or corn bagasse.

In some embodiments, the condensed phase comprises rice.

In some embodiments, the condensed phase comprises fruit.

In some embodiments, the condensed phase comprises citrus fruit.

In some embodiments, the condensed phase comprises stone fruit.

In some embodiments, the condensed phase comprises the wood of a stone fruit tree.

In some embodiments, the condensed phase comprises aggregate fruit.

In some embodiments, the condensed phase comprises berries.

In some embodiments, the condensed phase comprises drupes.

In some embodiments, the condensed phase comprises achenes.

In some embodiments, the condensed phase comprises pineapple.

In some embodiments, the condensed phase comprises tomato leaves.

In some embodiments, the condensed phase comprises dried vegetables.

In some embodiments, the condensed phase comprises dried peels.

In some embodiments, the condensed phase comprises dried citrus peels.

In some embodiments, the condensed phase comprises a species of ylang ylang.

In some embodiments, the condensed phase comprises spices.

In some embodiments, the condensed phase comprises herbs.

In some embodiments, the condensed phase comprises flowers.

In some embodiments, the condensed phase comprises seeds.

In some embodiments, the condensed phase comprises stems.

In some embodiments, the condensed phase comprises roots.

In some embodiments, the condensed phase comprises leaves.

In some embodiments, the condensed phase comprises rhizomes.

In some embodiments, the condensed phase comprises a fungus.

In some embodiments, the condensed phase comprises yeast.

In some embodiments, the condensed phase comprises dried yeast.

In some embodiments, the condensed phase comprises mushrooms.

In some embodiments, the condensed phase comprises algae.

In some embodiments, the condensed phase comprises bacteria.

In some embodiments, the condensed phase is comprised primarily of dried bacteria.

In some embodiments, the condensed phase comprises wood.

In some embodiments, the condensed phase comprises sawdust.

In some embodiments, the condensed phase comprises heartwood.

In some embodiments, the condensed phase comprises Amburana wood.

In some embodiments, the condensed phase comprises Amburana cearensis.

In some embodiments, the condensed phase comprises a species of sandalwood (Santalum).

In some embodiments, the condensed phase comprises Palo Santo wood (Bursera graveolens).

In some embodiments, the condensed phase comprises a species of oak (Quercus).

In some embodiments, the condensed phase comprises American oak (Quercus alba).

In some embodiments, the condensed phase comprises French oak (Quercus robur).

In some embodiments, the condensed phase comprises English oak (Quercus petraea).

In some embodiments, the condensed phase comprises Hungarian oak (Quercus frainetto).

In some embodiments, the condensed phase comprises mizunara oak (Quercus crispula).

In some embodiments, the condensed phase comprises Japanese oak (Quercus mongolica).

In some embodiments, the condensed phase comprises Quercus pedunculata.

In some embodiments, the condensed phase comprises Quercus sessiliflora.

In some embodiments, the condensed phase comprises apple wood.

In some embodiments, the condensed phase comprises cherry wood.

In some embodiments, the condensed phase comprises maple wood.

In some embodiments, the condensed phase comprises hickory wood.

In some embodiments, the condensed phase comprises mesquite wood.

In some embodiments, the condensed phase comprises pecan wood.

In some embodiments, the condensed phase comprises alder wood.

In some embodiments, the condensed phase comprises cypress wood.

In some embodiments, the condensed phase comprises cedar.

In some embodiments, the condensed phase comprises bourbon barrels, whiskey barrels, rum barrels, brandy barrels, wine barrels, madeira barrels, port barrels, tequila barrels, mescal barrels, sotol barrels, Cachaça barrels, or barrels that contained beer, wine, spirits, beverages or spices including, for example, sawdust obtained from any one or more of the foregoing.

In some embodiments, the condensed phase comprises coniferous wood. In some specific embodiments, the condensed phase comprises Araucaria; hoop pine (Araucaria cunninghamii); monkey puzzle tree (Araucaria araucana); Paraná pine (Araucaria angustifolia); cedar (Cedrus); celery-top pine (Phyllocladus aspleniifolius); cypress; Arizona cypress (Cupressus arizonica); bald cypress (Taxodium distichum); alerce (Fitzroya cupressoides); Hinoki cypress (Chamaecyparis obtusa); Lawson's cypress (Chamaecyparis lawsoniana); Mediterranean cypress (Cupressus sempervirens); Douglas fir (Pseudotsuga menziesii); European yew (Taxus baccata); fir (Abies); balsam fir (Abies balsamea); silver fir (Abies alba); noble fir (Abies procera); Pacific silver fir (Abies amabilis); hemlock (Tsuga); eastern hemlock (Tsuga canadensis); mountain hemlock (Tsuga mertensiana); western hemlock (Tsuga heterophylla); Huon pine (Lagarostrobos franklinii); kauri (Agathis australis); Queensland kauri (Agathis robusta); Japanese nutmeg-yew (Torreya nucifera); larch (Larix); European larch (Larix decidua); Japanese larch (Larix kaempferi); tamarack (Larix laricina); western larch (Larix occidentalis); pine (Pinus); European black pine (Pinus nigra); jack pine (Pinus banksiana); lodgepole pine (Pinus contorta); Monterey pine (Pinus radiata); Ponderosa pine (Pinus ponderosa); red pine (Pinus resinosa); Scots pine (Pinus sylvestris); white pine; eastern white pine (Pinus strobus); western white pine (Pinus monticola); sugar pine (Pinus lambertiana); southern yellow pine; loblolly pine (Pinus taeda); longleaf pine (Pinus palustris); pitch pine (Pinus rigida); shortleaf pine (Pinus echinata); red cedar; eastern red cedar (Juniperus virginiana); western red cedar (Thuja plicata); coast redwood (Sequoia sempervirens); rimu (Dacrydium cupressinum); spruce (Picea); Norway spruce (Picea abies); black spruce (Picea mariana); red spruce (Picea rubens); Sitka spruce (Picea sitchensis); white spruce (Picea glauca); sugi (Cryptomeria japonica); white cedar; northern white cedar (Thuja occidentalis); Atlantic white cedar (Chamaecyparis thyoides); African cypress (Widdringtonia species); pond cypress (Taxodium ascendens); Bald cypress (Taxodium distichum); Montezuma cypress (Taxodium mucronatum, Taxodium dubium); Chinese swamp cypress (Glyptostrobus pensilis); Cordilleran cypress (Austrocedrus chilensis); Cypress-pines; Cypress-pines (Callitris species); False cypress (Chamaecyparis species); Fujian cypress (Fokienia hodginsii); Guaitecas cypress (Pilgerodendron uviferum); Japanese cypress (Chamaecyparis obtusa); Patagonian cypress (Fitzroya cupressoides); Mediterranean cypress (Cupressus sempervirens); Monterey cypress (Cupressus macrocarpa); Nootka cypress (Cupressus nootkatensis); Siberian cypress (Microbiota decussata); Summer cypress (Bassia scoparia); Western red cedar (Thuja plicata); or nootka cypress (Cupressus nootkatensis).

In some embodiments, the condensed phase comprises angiosperm wood.

In some specific embodiments, the condensed phase comprises abachi (Triplochiton scleroxylon); acacia; African padauk (Pterocarpus soyauxii); afzelia (Afzelia africana); agba (Gossweilerodendron balsamiferum); alder (Alnus); black alder (Alnus glutinosa); red alder (Alnus rubra); ash (Fraxinus); black ash (Fraxinus nigra); blue ash (Fraxinus quadrangulata); common ash (Fraxinus excelsior); green ash (Fraxinus pennsylvanica); Oregon ash (Fraxinus latifolia); pumpkin ash (Fraxinus profunda); white ash (Fraxinus americana); aspen (Populus); bigtooth aspen (Populus gradidentata); European aspen (Populus tremula); quaking aspen (Populus tremuloides); Australian red cedar (Toona ciliata); ayan (Distemonanthus benthamianus); Agar wood; Aquilaria; balsa (Ochroma pyramidale); basswood; American basswood (Tilia americana); white basswood (Tilia heterophylla); American beech (Fagus grandifolia); birch (Betula); gray birch (Betula populifolia); black birch (Betula nigra); paper birch (Betula papyrifera); sweet birch (Betula lenta); yellow birch (Betula alleghaniensis); silver birch (Betula pendula); downy birch (Betula pubescens); blackbean (Castanospermum australe); blackwood; Australian blackwood (Acacia melanoxylon); African blackwood (Dalbergia melanoxylon); bloodwood (Brosimum rubescens); boxelder (Acer negundo); boxwood (Buxus sempervirens); Brazilian walnut (Ocotea porosa); brazilwood (Caesalpinia echinata); buckeye (Aesculus); horse-chestnut (Aesculus hippocastanum); Ohio buckeye (Aesculus glabra); yellow buckeye (Aesculus flava); butternut (Juglans cinerea); California bay laurel (Umbellularia californica); camphor tree (Cinnamomum camphora); cape chestnut (Calodendrum capense); catalpa (Catalpa); Ceylon satinwood (Chloroxylon swietenia); cherry (Prunus); black cherry (Prunus serotina); red cherry (Prunus pensylvanica); wild cherry (Prunus avium); chestnut (Castanea); chestnut (Castanea sativa); American chestnut (Castanea dentata); coachwood (Ceratopetalum apetalum); cocobolo (Dalbergia retusa); corkwood (Leitneria floridana); cottonwood; eastern cottonwood (Populus deltoides); swamp cottonwood (Populus heterophylla); cucumbertree (Magnolia acuminata); cumaru (Dipteryx); dogwood (Cornus); flowering dogwood (Cornus florida); Pacific dogwood (Cornus nuttallii); ebony (Diospyros); Andaman marblewood (Diospyros kurzii); ebène marbre (Diospyros melanida); African ebony (Diospyros crassiflora); Ceylon ebony (Diospyros ebenum); elm; American elm (Ulmus americana); English elm (Ulmus procera); rock elm (Ulmus thomasii); red elm (Ulmus rubra); wych elm (Ulmus glabra); eucalyptus; flooded gum (Eucalyptus grandis); white mahogany (Eucalyptus acmenoides); brown mallet (Eucalyptus astringens); southern mahogany (Eucalyptus botryoides); river red gum (Eucalyptus camaldulensis); karri (Eucalyptus diversicolor); blue gum (Eucalyptus globulus); rose gum (Eucalyptus grandis); york gum (Eucalyptus loxophleba); jarrah (Eucalyptus marginata); tallowwood (Eucalyptus microcorys); grey ironbark (Eucalyptus paniculata); blackbutt (Eucalyptus pilularis); mountain ash (Eucalyptus regnans); Australian oak (Eucalyptus obliqua); alpine ash (Eucalyptus delegatensis); red mahogany (Eucalyptus resinifera); swamp mahogany (Eucalyptus robusta); Sydney blue gum (Eucalyptus saligna); red ironbark (Eucalyptus sideroxylon); redwood (Eucalyptus transcontinentalis); Wandoo (Eucalyptus wandoo); European crabapple (Malus sylvestris); European pear (Pyrus communis); tigerwood (Astronium); greenheart (Chlorocardium rodiei); mpingo (Dalbergia melanoxylon); guanandi (Calophyllum brasiliense); gum (Eucalyptus); gumbo limbo (Bursera simaruba); hackberry (Celtis occidentalis); hickory (Carya); pecan (Carya illinoinensis); pignut hickory (Carya glabra); shagbark hickory (Carya ovata); shellbark hickory (Carya laciniosa); hornbeam (Carpinus); American hophornbeam (Ostrya virginiana); ipê (Handroanthus); African teak (Milicia excelsa); ironwood; balau (Shorea); American hornbeam (Carpinus caroliniana); sheoak (Casuarina equisetifolia); giant ironwood (Choricarpia subargentea); diesel tree (Copaifera langsdorffii); Borneo ironwood (Eusideroxylon zwageri); lignum vitae; guaiacwood (Guaiacum officinale); holywood (Guaiacum sanctum); takian (Hopea odorata); black ironwood (Krugiodendron ferreum); black ironwood (Olea); Lebombo ironwood (Androstachys johnsonii); Catalina ironwood (Lyonothamnus floribundus); Ceylon ironwood (Mesua ferrea); desert ironwood (Olneya tesota); Persian ironwood (Parrotia persica); Brazilian ironwood (Caesalpinia ferrea); yellow lapacho (Tabebuia serratifolia); jacarandá-boca-de-sapo (Jacaranda brasiliana); jacarandá de Brasil (Dalbergia nigra); jatobá (Hymenaea courbaril); kingwood (Dalbergia cearensis); lacewood; northern silky oak (Cardwellia sublimis); American sycamore (Platanus occidentalis); London plane (Platanus×acerifolia); limba (Terminalia superba); locust; black locust (Robinia pseudoacacia); honey locust (Gleditsia triacanthos); mahogany; genuine mahogany (Swietenia); West Indies mahogany (Swietenia mahagoni); bigleaf mahogany (Swietenia macrophylla); Pacific Coast mahogany (Swietenia humilis); African mahogany (Khaya); Chinese mahogany (Toona sinensis); Australian red cedar (Toona ciliata); Philippine mahogany (Toona calantas); Indonesian mahogany (Toona sureni); sapele (Entandrophragma cylindricum); sipo (Entandrophragma utile); tiama (Entandrophragma angolense); kosipo (Entandrophragma candollei); mountain mahogany (Entandrophragma caudatumi); Indian mahogany (Chukrasia velutina); Spanish Cedar (Cedrela odorata); light bosse (Guarea cedrata); dark bosse (Guarea thompsonii); American muskwood (Guarea grandifolia); carapa (Carapa guianensis); bead-tree (Melia azedarach); maple (Acer); hard maple; sugar maple (Acer saccharum); black maple (Acer nigrum); soft maple; boxelder (Acer negundo); red maple (Acer rubrum); silver maple (Acer saccharinum); European maple; sycamore maple (Acer pseudoplatanus); marblewood (Marmaroxylon racemosum); marri (Corymbia calophylla); meranti (Shorea); merbau (Intsia bijuga); mesquite; mopane (Colophospermum mopane); oak (Quercus); American oak or white oak (Quercus alba); bur oak (Quercus macrocarpa); post oak (Quercus stellata); swamp white oak (Quercus bicolor); southern live oak (Quercus virginiana); swamp chestnut oak (Quercus michauxii); chestnut oak (Quercus prinus); chinkapin oak (Quercus muhlenbergii); canyon live oak (Quercus chrysolepis); overcup oak (Quercus lyrata); red oak; northern red oak (Quercus rubra); eastern black oak (Quercus velutina); laurel oak (Quercus laurifolia); southern red oak (Quercus falcata); water oak (Quercus nigra); willow oak (Quercus phellos); Nuttall's oak (Quercus texana); okoumé (Aucoumea klaineana); olive (Olea europaea); pink ivory (Berchemia zeyheri); poplar; balsam poplar (Populus balsamifera); black poplar (Populus nigra); hybrid black poplar (Populus×canadensis); purpleheart (Peltogyne); a species in the genus Prosopis; Queensland maple (Flindersia brayleyana); Queensland walnut (Endiandra palmerstonii); ramin (Gonystylus); redheart, chakté-coc (Erythroxylon mexicanum); sal (Shorea robusta); sweetgum (Liquidambar styraciflua); sandalwood (Santalum); Australian sandalwood (Santalum spicatum); Indian sandalwood (Santalum album); Hawaiian sandalwood (Santalum ellipticum, Santalum freycinetianum, Santalum paniculatum, Santalum haleakalae); Santalum acuminatum; Santalum yasi; Santalum spicatum; sassafras (Sassafras albidum); southern sassafras (Atherosperma moschatum); satiné (acuminatumBrosimum rubescens); silky oak (Grevillea robusta); silver wattle (Acacia dealbata); sourwood (Oxydendrum arboreum); Spanish-cedar (Cedrela odorata); Spanish elm (Cordia alliodora); tamboti (Spirostachys africana); teak (Tectona grandis); Thailand rosewood (Dalbergia cochinchinensis); tupelo (Nyssa); black tupelo (Nyssa sylvatica); tulip tree (Liriodendron tulipifera); turpentine (Syncarpia glomulifera); walnut (Juglans); Eastern black walnut (Juglans nigra); common walnut (Juglans regia); wenge (Millettia laurentii); panga-panga (Millettia stuhlmannii); willow (Salix); black willow (Salix nigra); cricket-bat willow (Salix alba Caerulea); white willow (Salix alba); weeping willow (Salix babylonica); or zingana (Microberlinia brazzavillensis).

In some specific embodiments, the condensed phase comprises Amburana; Amburana acreana; Amburana cearensis; Amburana erythrosperma; Apple; Malus domestica; Malus sieversii; Palo Santo (Bursera graveolens); clove (Syzygium aromaticum); star anise (Illicium verum); cinnamon; Ceylon cinnamon (Cinnamomum verum); Cinnamomum burmannii; Cinnamomum cassia; Cinnamomum loureiroi; Cinnamomum citriodorum; Brazilian rosewood (Dalbergia nigra); cocobolo (Dalbergia retusa); lignum vitae (Guaiacum officinale); raspberry jam wood (Acacia acuminata); Torreya; Torreya californica; Torreya fargesii; Torreya grandis; Torreya jackii; Torreya nucifera; Torreya taxifolia; thuya (Tetraclinis articulata); cacao wood (Theobroma cacao); peach (Prunus persica); apricot (Prunus armeniaca; Prunus brigantina; Prunus cathayana; Prunus dasycarpa; Prunus hongpingensis; Prunus hypotrichodes; Prunus limeixing; Prunus mandshurica; Prunus mume; Prunus sibirica; Prunus zhengheensis); plum (Prunus domestica; Prunus salicina; Prunus simonii); almond; Prunus amygdalus; Prunus dulcis; pistachio (Pistacia vera); honey mesquite (Prosopis glandulosa); velvet mesquite (Prosopis velutina); mesquite (Prosopis spp, Prosopis pallida or Prosopis juliflora); creosote bush (Larrea tridentata); mulberry (Morus); crabapple (Malus sylvestris); or a species of magnolia (Magnoliaceae).

In some embodiments, the condensed phase comprises biomass of a mute plant. In some specific embodiments, the mute plant is lily of the valley (Convallaria), lilac (Syringa), honeysuckle (Lonicera), violet (Violaceae), seringa (Philadephaceae), hyacinth (Hyacinthus), sweet pea (Lathyrus), or a species of magnolia (Magnoliaceae) flower. In some very specific embodiments, the condensed phase comprises biomass of lily of the valley.

In some embodiments, the condensed phase comprises plum blossoms, cherry blossoms, apple blossoms, orange blossoms, lemon blossoms, lime blossoms, satsuma blossoms, osmanthus blossoms, jasmine blossoms, Frangipani (Plumeria) blossoms, Nyctanthes arbor-tristis, lavender, tuberose flowers, lilies, rose, rose blossoms, ylang ylang (Cananga odorata), Manoranjitham (Artabotrys hexapetalus), Narcissus flowers, Scented Primrose (Primula vulgaris), Sweet Autumn Clematis (Clematis terniflora), Nicotiana (Nicotiana), Viburnum, Mock Orange (Philadelphus), Lilac (Syringa), Angel's Trumpet (Brugmansia), Daphne, Night Scented Stocks (Matthiola longipetala), Magnolia, Brunfelsia pauciflora, Freesia, Wrightia religiosa, Hedychium coronarium, Fagraea berteroana, Tabernaemontana divaricate, a species of magnolia (Magnoliaceae), Magnolia champaca, Cestrum nocturnum, Gardenia, Wisteria, Azalia, Sweet osmanthus, Camellia, Sasanqua Camellia, or Magnolia grandiflora.

In some embodiments, the condensed phase comprises Agarwood: oud; Aquilaria; Aquilaria malaccensis; frankincense; Boswellia; Boswellia sacra; Boswellia bhaw-dajiana; Boswellia carteri; Boswellia frereana, Boswellia serrata; Boswellia thurifera; Boswellia papyrifera; Galbanum; Ferula; Ferula gummosa; Ferula rubricaulis; orris; Rhizoma iridis; Iris germanica; Iris pallida; a species in the genus Iridaceae; lis root, iris root; Amber; Baltic Amber; Ambergris; Ambrette; Ambrette seeds; Amyris; Balsamic; benzoin; pine; resin; juniper; turpentine; Styrax tree; Bergamot; bergamot orange; Clone; Cashmeran; galbanum resin; Guaiac Wood; Hedione; Heliotrope; flowers from the heliotropism family; vetiver; vetiver roots; patchouli; patchouli leaves; patchouli bush; Indole, Iso-E-Super, Jasmine; Coconut; Labdanum; rockrose bush; Leather; Myrrh; Commiphora; Narcissus; Oakmoss; lichen; Opopanax; Sweet Myrrh; balsam; Osmanthus; Rose; Clary sage; or Tonka bean.

In some embodiments, the condensed phase comprises allspice (Pimenta dioica); angelica (Angelica archangelica); anise (Pimpinella anisum); asafoetida (Ferula assa-foetida); bay leaf (Laurus nobilis); basil (Ocimum basilicum); bergamot (Monarda species); black cumin (Nigella sativa); black mustard (Brassica nigra); black pepper (Piper nigrum); borage (Borago officinalis); brown mustard (Brassica juncea); burnet (Sanguisorba minor and S. officinalis); caraway (Carum carvi); cardamom (Elettaria cardamomum); cassia (Cinnamomum cassia); catnip (Nepeta cataria); cayenne pepper (Capsicum annuum); celery seed (Apium graveolens, variety dulce); chervil (Anthriscus cerefolium); chicory (Cichorium intybus); chili pepper (Capsicum species); chives (Allium schoenoprasum); cicely (Myrrhis odorata); cilantro (Coriandrum sativum); cinnamon (Cinnamomum verum); clove (Syzygium aromaticum); coriander (Coriandrum sativum); costmary (Tanacetum balsamita); cumin (Cuminum cyminum); curry; dill (Anethum graveolens); fennel (Foeniculum vulgare); fenugreek (Trigonella foenum-graecum); filé (Sassafras albidum); ginger (Zingiber officinale); grains of paradise (Aframomum melegueta); holy basil (Ocimum tenuiflorum); horehound (Marrubium vulgare); horseradish (Armoracia rusticana); hyssop (Hyssopus officinalis); lavender (Lavandula species); lemon balm (Melissa officinalis); lemon grass (Cymbopogon citratus); lemon verbena (Aloysia citrodora); licorice (Glycyrrhiza glabra); lovage (Levisticum officinale); mace (Myristica fragrans); marjoram (Origanum majorana); nutmeg (Myristica fragrans); oregano (Origanum vulgare); paprika (Capsicum annuum);

parsley (Petroselinum crispum); peppermint (Mentha×piperita); poppy seed (Papaver somniferum); rosemary (Salvia rosmarinus); rue (Ruta graveolens); saffron (Crocus sativus); sage (Salvia officinalis); savory (Satureja hortensis and S. montana); sesame (Sesamum indicum); sorrel (Rumex species); star anise (Illicium verum); spearmint (Mentha spicata); tarragon (Artemisia dracunculus); thyme (Thymus vulgaris); turmeric (Curcuma longa); vanilla (Vanilla planifolia and V. tahitensis); wasabi (Eutrema japonicum); or white mustard (Sinapis alba).

In some embodiments, the condensed phase comprises saffron.

In some embodiments, the condensed phase comprises vanilla.

In some embodiments, the condensed phase comprises cinnamon.

In some embodiments, the condensed phase comprises capsaicin.

In some embodiments, the condensed phase comprises menthol.

In some embodiments, the condensed phase comprises a type of peppercorn.

In some embodiments, the condensed phase comprises leather.

In some embodiments, the condensed phase comprises a plant species from the genus Nicotiana.

In some embodiments, the condensed phase comprises Nicotiana acuminata; Nicotiana Africana; Nicotiana alata; Nicotiana attenuata; Nicotiana benthamiana; Nicotiana clevelandii; Nicotiana glauca; Nicotiana glutinosa; Nicotiana langsdorffii; Nicotiana longiflora; Nicotiana occidentalis; Nicotiana obtusifolia; Nicotiana otophora; Nicotiana plumbaginifolia; Nicotiana quadrivalvis; Nicotiana rustica; Nicotiana suaveolens; Nicotiana sylvestris; Nicotiana tabacum; Nicotiana tomentosiformis; Nicotiana×didepta; Nicotiana debneyi×Nicotiana. tabacum; Nicotiana×digluta; Nicotiana glutinosa×Nicotiana tabacum; Nicotiana×sanderae; or Nicotiana alata×Nicotiana forgetiana.

In some embodiments, the condensed phase comprises tea.

In some embodiments, the condensed phase comprises one or more of the following types of tea: Green tea; Chun Mee; Chun Lu; Bi Luo Chun; Gunpowder; Maofeng; Yellow; Jasmine; Anji Bai Cha; Maojian; Taiping Houkui; Jin Shan; Longjing (Dragon Well); Sejak; Ujeon; Jungjak; Daejak; Sencha; Gyokuro; Kabusecha; Tencha; Matcha; Mecha; Shincha; Hojicha; Kukicha; Bancha; Genmaicha; Konacha; Kamairicha; Tamaryokucha; Black tea; Assam;

English Breakfast; Earl Grey; Darjeeling; Rukeri; Pu-Erh; Scottish Afternoon; Irish Breakfast; Milima; Ceylon; Chai; Panyang Congou; Keemun; Lapsang Souchong; Golden Tips; Temi Sikkim; Nimbu; Wakuocha; White tea; Silver Needle; White Peony; Shou Mei; Gong Mei; Darjeeling White; Oolong tea; Da Hong Pao; Shui Jin Gui; Tie Luo Han; Shui Xian; Bai Jiguan; Tieguanyi (Iron Goddess); Mi Lan Xiang Dan Con; Ancient Tree Dan Cong; Guan Yin; Dancong; Cassia; Da Yu Lin; Dong Ding; Dong Fang Meiren; Alishan; Pouchong; Ruan Zhi; Jin Xuan; or Li Shan.

In some embodiments, the condensed phase comprises a herbal tea.

In some embodiments, the condensed phase comprises one or more of the following types of tea Avocado Leaf; Bamboo; Butterfly Pea Flower; Chaga Mushroom; Chamomile; Lavender; Liquorish; Guayusa; Honeysuckle Flower; Lemon; Mint; Olive Leaves; Hibiscus; Rooibos; Turmeric; Pumpkin Spice; Chrysanthemum; Buckwheat; Honeybush; Bush; Mamaki; Yaupon; or Yerba mate.

In some embodiments, the condensed phase comprises yerba mate.

In some embodiments, the condensed phase comprises rooibos.

In some embodiments, the condensed phase comprises meat or dried meat.

In some embodiments, the condensed phase comprises dried mushrooms.

In some embodiments, the condensed phase comprises beans.

In some embodiments, the condensed phase comprises soybeans.

In some embodiments, the condensed phase comprises fermented plants.

In some embodiments, the condensed phase comprises dried fermented plants. In some embodiments, the boiling point of the molecule at the pressure of the gas phase is at least 10 degrees Celsius greater than the temperature of the gas phase. In some specific embodiments, the boiling point of the molecule at the pressure of the gas phase is at least 20 degrees Celsius greater than the temperature of the gas phase. In some even more specific embodiments, the boiling point of the molecule at the pressure of the gas phase is at least 40 degrees Celsius greater than the temperature of the gas phase. In some very specific embodiments, the boiling point of the molecule at the pressure of the gas phase is at least 100 degrees Celsius greater than the temperature of the gas phase.

Various aspects of the disclosure relate to a method to separate a molecule from an impurity, comprising: providing a composition comprising the molecule and the impurity, wherein the molecule is present in the composition in a solid phase or a liquid phase, and the impurity is present in the composition in a solid phase or a liquid phase; converting the molecule into a vaporized molecule in a gas phase, wherein the gas phase has a pressure and a temperature, the molecule has a boiling point at the pressure and a vapor pressure at the temperature, the pressure of the gas phase is greater than the vapor pressure of the molecule, the boiling point of the molecule is greater than the temperature of the gas phase, and either the impurity lacks a vapor pressure or the impurity has a vapor pressure at the temperature that is less than the vapor pressure of the molecule at the temperature; separating the vaporized molecule from the impurity; and condensing the vaporized molecule into a condensed molecule.

The following examples provide a framework to implement certain aspects of the disclosure, and these examples do not limit the scope of this patent document or any claim that matures from the disclosure of this patent document.

### Example 1: Extraction of Coumarin from Amburana Wood Barrels.

The wood of Amburana cearensis is known to contain coumarin. Amburana wood barrels are commonly used to age Cachaça, a distilled spirit that has similar qualities to rum. Coumarin is the primary source of aroma in Amburana wood, but it is only present in small quantities, making it exceptionally difficult to extract using conventional solvent methods.

Three kilograms of medium-toast Amburana cearensis wood was ground and extracted using an extraction machine as described in European U.S. Pat. No. 3,283,606B1 . The extraction machine was operated with an extraction temperature that did not exceed 205 degrees Celsius. The vaporized coumarin was collected in three liters of ethanol. 100 milliliters of the resulting extract was analyzed using high-pressure liquid chromatography (HPLC). The extract was found to contain 1,138 milligrams of coumarin per kilogram.

Based on known quantities of coumarin in toasted Amburana cearensis, the method of the present disclosure efficiently extracted coumarin at an extraction temperature far below its known boiling point. Coumarin has a boiling point of 301.7 degrees Celsius, yet the extraction machine never exceeded an extraction temperature of 205 degrees Celsius.

The foregoing results confirm that a composition of this disclosure was produced in the extraction machine, in which the condensed phase consisted of the ground Amburana cearensis wood, the gas phase comprised the gas stream of the extraction machine, and the molecule was coumarin.

### Example 2: Extraction of Toasted French Oak

Toasted French oak is known to contain a just few milligrams per kilogram of vanillin, eugenol, and isoeugenol, yet these compounds make a profound contribution to barrel-aged whiskey and other barrel-aged spirits.

Three kilograms of medium-toast French oak was ground and extracted using an extraction machine as described in European U.S. Pat. No. 3,283,606B1 . The extraction machine was operated at an extraction temperature that did not exceed 205 degrees Celsius. The vaporized vanillin, eugenol, and isoeugenol was collected in three kilograms of a collection solvent consisting of ethanol and water. 100 milliliters of the resulting extract was analyzed using a gas chromatography mass spectrometer (GC-MS) and found to contain 15.8 milligrams of vanillin per kilogram, 0.2 milligrams of eugenol per kilogram, and 0.9 milligrams of isoeugenol per kilogram.

Based on the known quantities of vanillin, eugenol, and isoeugenol in toasted French oak, the method of the present disclosure efficiently extracted each of these compounds at temperatures far below their known boiling points. Vanillin has a boiling point of 285 degrees Celsius, yet the extraction machine never exceeded an extraction temperature of 205 degrees Celsius. Eugenol has a boiling point of 252 degrees Celsius, yet the extraction machine never exceeded an extraction temperature of 205 degrees Celsius. Isoeugenol has a boiling point of 266 degrees Celsius, yet the extraction machine never exceeded an extraction temperature of 205 degrees Celsius.

The foregoing results confirm that a composition of this disclosure was produced in the extraction machine, in which the condensed phase consisted of the ground French oak, the gas phase comprised the gas stream of the extraction machine, and the molecule was either vanillin, eugenol, or isoeugenol.

### Example 3: Extraction of Toasted American Oak

Toasted American oak is known to contain a just few milligrams per kilogram of vanillin, eugenol, and isoeugenol, yet these compounds make a profound contribution to barrel-aged whiskey and other barrel-aged spirits.

Three kilograms of dark-toast American oak was ground and extracted using an extraction machine as described in European U.S. Pat. No. 3,283,606B1 . The extraction machine was operated with an extraction temperature that did not exceed 205 degrees Celsius. The vaporized vanillin, eugenol, and isoeugenol was collected in three kilograms of a collection solvent consisting of ethanol and water. The resulting extract was analyzed using GC-MS methods and was found to contain 13.1 milligrams of vanillin per kilogram, 0.9 milligrams of eugenol per kilogram, and 2.4 milligrams of isoeugenol per kilogram.

Based on the known quantities of these compounds in toasted American oak, the method of the present disclosure efficiently extracted each of these compounds at temperatures far below their known boiling points. Vanillin has a boiling point of 285 degrees Celsius, yet the extraction machine never exceeded an extraction temperature of 205 degrees Celsius. Eugenol has a boiling point of 252 degrees Celsius, yet the extraction machine never exceeded an extraction temperature of 205 degrees Celsius. Isoeugenol has a boiling point of 266 degrees Celsius, yet the extraction machine never exceeded an extraction temperature of 205 degrees Celsius.

The foregoing results confirm that a composition of this disclosure was produced in the extraction machine, in which the condensed phase consisted of the ground American oak, the gas phase comprised the gas stream of the extraction machine, and the molecule was either vanillin, eugenol, or isoeugenol.

### Example 4: Extraction of Sandalwood

Sandalwood derives its signature scent from alpha-santalol, which boils at 302 degrees Celsius.

Three kilograms of sandalwood is finely ground to increase its surface area. The ground sandalwood is homogenized by mixing to ensure consistency throughout. A randomized 100-gram sample of the homogenized sandalwood is tested using HPLC to analyze its starting alpha-santalol content. The test results indicate that the pre-extraction sandalwood contains approximately 33,000 milligrams of alpha-santalol per kilogram of sandalwood.

The remaining 2,900 grams of sandalwood is extracted using the method of the present disclosure at a sweep gas temperature of 210 degrees Celsius and a pressure of 760.00 mm Hg. The vaporized alpha-santalol is captured in three kilograms of a collection solvent consisting of ethanol and water to produce a highly aromatic sandalwood extract.

100 milliliters of the extract is homogenized and tested using HPLC methods. The test results indicate that the extract contains 23,430 milligrams per kilogram. The depleted post-extraction sandalwood is homogenized by mixing, and a 100-gram sample is tested using HPLC methods. The test results indicate that the post-extraction sandalwood contains only 7,260 milligrams of alpha-santalol per kilogram of sandalwood.

Alpha-santalol has a boiling point of 302 degrees Celsius, yet efficient extraction is achieved at 210 degrees Celsius. A comparison of the HPLC results for the pre-extraction sandalwood and the post-extraction sandalwood indicates that approximately 78% of the available alpha-santalol has been removed from the sandalwood. A comparison of the HPLC results for the pre-extraction sandalwood and the extract indicates that approximately 76% of the available alpha-santalol is captured from the sandalwood. Approximately 2% of the total alpha-santalol is unaccounted for.

The foregoing results confirm that a composition of this disclosure is produced in the extraction machine, in which the condensed phase consists of the ground sandalwood, the gas phase comprises the gas stream of the extraction machine, and the molecule is alpha-santalol.

### Example 5: Extraction of Sandalwood

Three kilograms of ground sandalwood were ground and extracted using the methods of the present invention with a sweep gas temperature of 200 degrees Celsius. A highly aromatic sandalwood extract was produced that was judged by several senior fragrance industry experts to have greater aromatic intensity than steam-distilled and solvent-extracted reference samples. A sensory panel was conducted on the sandalwood source material before and after extraction using the method of the present disclosure. The starting source material displayed a strong aroma of freshly ground sandalwood. The extracted source material possessed little remaining aroma. This experiment was repeated at a sweep gas temperature of 170 Celsius, 180 Celsius, and 210 Celsius. In all cases, most of the aroma associated with alpha-santalol was stripped from the starting source material.

The foregoing results confirm that a composition of this disclosure was produced in the extraction machine, in which the condensed phase consisted of the ground sandalwood, the gas phase comprised the gas stream of the extraction machine, and the molecule was alpha-santalol.

### Example 6: Extraction of Orris

Orris root derives its signature scent from irone, which boils at 295 degrees Celsius.

Three kilograms of orris is finely ground to increase its surface area. The ground orris is homogenized by mixing to ensure consistency throughout. A randomized 100-gram sample of the homogenized orris is tested using HPLC methods to analyze its starting irone content. The test results indicate that the pre-extraction orris contains approximately 1,880 milligrams of irone per kilogram of orris.

The remaining 2,900 grams of orris is extracted using the method of the present disclosure at a sweep gas temperature of 200 degrees Celsius and a pressure of 760.00 mm Hg. The vaporized irone is captured in three kilograms of a collection solvent consisting of ethanol and water to produce a highly aromatic orris extract.

100 milliliters of the extract is tested using HPLC methods. The test results indicate that the extract contains 1,523 milligrams of irone per kilogram. The depleted post-extraction orris is homogenized by mixing, and a 100-gram sample is tested using HPLC methods. The test results indicate that the post-extraction orris contains only 301 milligrams of irone per kilogram of orris.

Irone has a boiling point of 295 degrees Celsius, yet efficient extraction is achieved at 200 degrees Celsius. A comparison of the HPLC results for the pre-extraction orris and the post-extraction orris indicates that approximately 84% of the available irone has been removed from the orris. A comparison of the HPLC results for the pre-extraction orris and the extract indicates that approximately 81% of the available irone is captured from the orris. Approximately 3% of the total irone is unaccounted for.

The foregoing results confirm that a composition of this disclosure is produced in the extraction machine, in which the condensed phase consists of the ground orris, the gas phase comprises the gas stream of the extraction machine, and the molecule is irone.

### Example 7: Extraction of Orris

Three experiments were performed using ground orris as a source material. For each experiment, three kilograms of ground orris root was extracted using the methods of the present invention. In the first experiment, a sweep gas temperature of 170 degrees Celsius was utilized. In the second experiment a sweep gas temperature of 190 degrees Celsius was utilized. In the third experiment, a sweep gas temperature of 205 degrees Celsius was utilized. In each case, a highly aromatic orris extract was produced. Several senior fragrance industry experts determined that the orris extract produced with the methods of the present disclosure were preferred over steam-distilled and solvent-extracted reference samples. For each experiment, a sensory panel was conducted on the source material before and after extraction using the method of the present disclosure. The starting source material displayed a strong aroma of freshly-ground orris. For all experiments, the extracted source material was found to possess little remaining aroma, with only slightly more aroma present in the 170 degrees Celsius extracted source material compared to the 205 degrees Celsius extracted source material. In each case, the vast majority of the irone present in the orris had apparently been removed from the source material and deposited into the extract.

The foregoing results confirm that a composition of this disclosure was produced in the extraction machine, in which the condensed phase consisted of the ground orris, the gas phase comprised the gas stream of the extraction machine, and the molecule was irone.

### Example 8: Extraction of Various Woods and Detection of High-Boiling Point Compounds

Several different compositions consisting of different types of wood, wood barrels that formerly contained distilled spirits, and wood barrels that formerly contained wine, were each separately ground into sawdust particles measuring less than 1 millimeter in length on average. In a series of separate tests, the wood particles were extracted using methods of this disclosure, and GC-MS analyses were performed on the resulting extracts. The experiments were performed in an extraction machine as described in European U.S. Pat. No. 3,283,606B1 . For each test, the sweep gas was heated to approximately 205 degrees Celsius. The wood particles were introduced continuously to an extraction chamber of the extraction machine at a metered rate by an auger. Upon entering the extraction chamber, the wood particles were bombarded with the sweep gas. The bombardment of the sweep gas aerosolized the wood particles and transported the wood particles through the extraction chamber along with the sweep gas. The aerosolized composition remained in contact with the sweep gas for several seconds as it passed through the length of the extraction chamber. The extraction chamber included turns, to create turbulent air flow to increase the mass transfer rate of molecules of the composition into vaporized molecules. In each test, molecules were evaporated from each of the different compositions to form a vapor. A cyclone separator was used to separate the vaporized molecules evaporated vapor from the non-evaporated components of the wood particles. The sweep gas containing the separated vaporized molecules was passed through a spray of collection solvent to condense the molecules into condensed molecules. The collection solvent contained a blend of ethanol and water to attract, absorb and hold the molecules. To reach the desired concentration of condensed molecules in the collection solvent, in each test, the spray of collection solvent was continuously recirculated by a liquid pump, and the sweep gas containing vaporized molecules was passed continuously passed through the spray of collection solvent. The embodiment was operated continuously until approximately three kilograms of wood particles had passed through the extraction machine. In each case, an aromatic wood extract was captured in the collection solvent. 100 milliliters of each extract was analyzed using GC-MS and HPLC methods. Even though the sweep gas and extraction chamber were held at or below approximately 205 degrees Celsius, significant quantities of the following higher-boiling-point molecules were detected in the different tests: furyl-hydroxymethyl ketone (CAS: 17678-19-2), which boils a 239 degrees Celsius; 2,5-furandicarboxaldehyde (CAS: 823-82-5) which boils at 276 to 277 degrees Celsius; nonan-1-ol (CAS: 143-08-8), which boils at 214 degrees Celsius; decan-1-ol (CAS: 112-53-8), which boils at 231 degrees Celsius; dodec-1-ol (CAS: 112-53-8), which boils at 230 degrees Celsius; tetradecane-1-ol (CAS: 112-72-1), which boils at 289 degrees Celsius; hexadecane-1-ol (CAS: 36653-82-4), which boils at 344 degrees Celsius; octadecane-1-ol (CAS: 112-92-5) which boils at 384 degrees Celsius; 2-decen-1-ol (CAS: 22104-80-9) which boils at 229 degrees Celsius; phenylethyl alcohol (CAS: 60-12-8), which boils at 219-221 degrees Celsius; tetradecanal (CAS: 124-25-4), which boils at 260 degrees Celsius; (Z)-2-decenal (CAS: 2497-25-8), which boils at 226-230 degrees Celsius; (E, E)-2,4-decadienal (CAS: 25152-84-5), which boils at 279 to 280 degrees Celsius; octanoic acid (CAS: 124-07-2), which boils at 237 degrees Celsius; nonanoic acid (CAS: 112-05-0), which boils at 254 degrees Celsius; n-decanoic acid (CAS: 334-48-5), which boils at 268.00 to 270.00 degrees Celsius; n-hexadecanoic acid (CAS: 57-10-3), which boils at 351 degrees Celsius; heptadecanoic acid (CAS: 506-12-7), which boils at 263 degrees Celsius; octadecanoic acid (CAS: 57-11-4), which boils at 361degrees Celsius; 2-ethylhexanoic acid (CAS: 149-57-5), which boils at 228 degrees Celsius; trans-2-undecenoic acid (CAS: 15790-94-0), which boils at 295 degrees Celsius; benzoic acid (CAS: 65-85-0), which boils at 249 degrees Celsius; phthalic acid (CAS: 88-99-3), which boils at 289 degrees Celsius; methyl nonanoate (CAS: 1731-84-6), which boils at 213 degrees Celsius; octyl butanoate (CAS: 110-39-4), which boils at 224 degrees Celsius; isopropyl myristate (CAS: 110-27-0), which boils at 315 degrees Celsius; ethyl nicotinate (CAS: 614-18-6), which boils at 224 degrees Celsius; 3-hexenyl butanoate (CAS: 53398-84-8), which boils at 213 degrees Celsius; diethyl butanedioate (CAS: 123-25-1), which boils at 217 degrees Celsius; diethyl itaconate (CAS: 2409-52-1), which boils at 213 degrees Celsius; benzyl benzoate (CAS 120-51-4), which boils at 323 degrees Celsius; methyl salicylate (CAS: 119-36-8), which boils at 222-224 degrees Celsius; eugenol (CAS 97-53-0), which boils at 252-253 degrees Celsius; vanillin (CAS 121-33-5), which boils at 285-286 degrees Celsius; (Z)-isoeugenol (CAS: 5932-68-3), which boils at 266-268 degrees Celsius; ethyl anisate (CAS: 94-30-4), which boils at 263 degrees Celsius; 4-methylguauacol (CAS: 93-51-6), which boils at 221 degrees Celsius; (E)-2,6-dimethoxy-4-(prop-1-en-1-yl)phenol (CAS: 20675-95-0), which boils at 305 degrees Celsius; thymol (CAS: 89-83-8), which boils at 232 degrees Celsius; caryophyllene (CAS: 87-44-5), which boils at 256-259 degrees Celsius; α-bisabolol (CAS 515-69-5), which boils at 314-315 degrees Celsius; (4R,5R)-5-butyl-4-methyloxolan-2-one (CAS: 55013-32-6), which boils at 245-247 degrees Celsius; (4S,5R)-5-butyl-4-methyloxolan-2-one (CAS: 39638-67-0), which boils at 246 degrees Celsius; β-ionone (CAS: 8013-90-9), which boils at 255 degrees Celsius; δ-tetradecalactone (CAS: 2721-22-4), which boils at 322 degrees Celsius; bis(2-furfuryl)disulfide (CAS: 4437-20-1), which boils at 229-230 degrees Celsius; hydrocoumarin (CAS: 119-84-6), which boils at 272 degrees Celsius; and coumarin (CAS: 91-64-5), which boils at 301.7 degrees Celsius. In most cases, the mass extracted of the above molecules represented corresponded to a majority of the mass known to present in the starting wood compositions. Several hundred other compounds were also found in testing that have not been mentioned.

The foregoing results confirm that a composition of this disclosure was produced in the extraction machine, in which the condensed phase consisted of the ground wood, the gas phase comprised the gas stream of the extraction machine, and the molecule was any one of the molecules described in the preceding paragraph.

What is claimed is:

1. A composition, comprising a gas phase and a condensed phase, wherein:
the condensed phase comprises sawdust;
the gas phase comprises a molecule;
the molecule is furfural;
the condensed phase comprises the molecule;
the gas phase has a temperature and a pressure;
the temperature of the gas phase is greater than 100 degrees Celsius and no greater than 235 degrees Celsius;
the composition has a rate of sensible heat transfer from the gas phase to the condensed phase;
the molecule has a boiling point at the pressure of the gas phase;
the boiling point of the molecule is greater than the temperature of the gas phase;
the molecule has a vapor pressure at the temperature of the gas phase;
the vapor pressure of the molecule is less than the pressure of the gas phase;
the condensed phase consists of one or both of a solid phase and a liquid phase;
the condensed phase is suspended in the gas phase;
the composition comprises at least 1 gram of the condensed phase per cubic meter of the gas phase;
the composition comprises no greater than 1 gram of the condensed phase per liter of the gas phase;
the composition comprises at least 2 micrograms of the furfural per gram of the condensed phase;
the composition comprises no greater than 200 milligrams of the furfural per gram of the condensed phase;
the composition comprises at least 2 micrograms of the furfural per gram of the gas phase;
the composition comprises no greater than 200 milligrams of the furfural per gram of the gas phase;
the composition has an average velocity of at least 100 millimeters per second;
the composition has a turbulent flow; and
the condensed phase has a surface-area-to-volume ratio of at least 500 per meter.

2. A composition, comprising a gas phase and a condensed phase, wherein:
the condensed phase comprises sawdust;
the gas phase comprises a molecule;
the molecule is furfural;
the condensed phase comprises the molecule;
the gas phase has a temperature and a pressure;
the temperature of the gas phase is greater than 100 degrees Celsius and no greater than 235 degrees Celsius;
the molecule has a boiling point at the pressure of the gas phase;
the boiling point of the molecule is greater than the temperature of the gas phase;
the molecule has a vapor pressure at the temperature of the gas phase;
the vapor pressure of the molecule is less than the pressure of the gas phase;
the condensed phase consists of one or both of a solid phase and a liquid phase;
the condensed phase is suspended in the gas phase;
the composition comprises at least 1 gram of the condensed phase per cubic meter of the gas phase;
the composition comprises no greater than 1 gram of the condensed phase per liter of the gas phase;
the composition comprises at least 2 micrograms of the furfural per gram of the condensed phase;
the composition comprises no greater than 200 milligrams of the furfural per gram of the condensed phase;
the composition comprises at least 2 micrograms of the furfural per gram of the gas phase;
the composition comprises no greater than 200 milligrams of the furfural per gram of the gas phase;
the composition has an average velocity of at least 100 millimeters per second;
the composition has a turbulent flow; and
the condensed phase has a surface-area-to-volume ratio of at least 500 per meter.

3. A composition, comprising a gas phase and a condensed phase, wherein:
the condensed phase comprises sawdust;
the gas phase comprises a molecule;
the molecule is furfural;
the condensed phase comprises the molecule;
the gas phase has a temperature and a pressure;
the temperature of the gas phase is greater than 100 degrees Celsius and no greater than 235 degrees Celsius;
the molecule has a boiling point at the pressure of the gas phase;
the boiling point of the molecule is greater than the temperature of the gas phase;
the molecule has a vapor pressure at the temperature of the gas phase;
the vapor pressure of the molecule is less than the pressure of the gas phase;
the condensed phase consists of one or both of a solid phase and a liquid phase;
the condensed phase is suspended in the gas phase;
the composition comprises at least 1 gram of the condensed phase per cubic meter of the gas phase;
the composition comprises at least 2 micrograms of the furfural per gram of the condensed phase;
the composition comprises at least 2 micrograms of the furfural per gram of the gas phase;
the composition has an average velocity of at least 100 millimeters per second;
the composition has a turbulent flow; and
the condensed phase has a surface-area-to-volume ratio of at least 500 per meter.

4. The composition of claim 3, wherein:
the composition has a rate of sensible heat transfer from the gas phase to the condensed phase of at least 2 joules per gram of the condensed phase per second and no greater than 20 kilojoules per gram of the condensed phase per second;
the composition comprises no greater than 1 gram of the condensed phase per liter of the gas phase;
the composition comprises no greater than 200 milligrams of the furfural per gram of the gas phase;
the composition has a vaporization rate for conversion of the molecule in the condensed phase into the molecule in the gas phase;
the composition has a condensation rate for conversion of the molecule in the gas phase into the molecule in the condensed phase;
the composition has a mass transfer rate, which is equal to the vaporization rate minus the condensation rate; and
the mass transfer rate is a positive number.

5. The composition of claim 3, wherein:
the composition has a rate of sensible heat transfer from the gas phase to the condensed phase of at least 2 joules per gram of the condensed phase per second;
the composition comprises no greater than 1 gram of the condensed phase per liter of the gas phase;
the composition comprises no greater than 200 milligrams of the furfural per gram of the gas phase;
the composition has a vaporization rate for conversion of the molecule in the condensed phase into the molecule in the gas phase;
the composition has a condensation rate for conversion of the molecule in the gas phase into the molecule in the condensed phase;
the composition has a mass transfer rate, which is equal to the vaporization rate minus the condensation rate; and
the mass transfer rate is a positive number.

6. The composition of claim 3, wherein:
the composition has a rate of sensible heat transfer from the gas phase to the condensed phase of no greater than 20 kilojoules per gram of the condensed phase per second;
the composition comprises no greater than 1 gram of the condensed phase per liter of the gas phase;
the composition comprises no greater than 200 milligrams of the furfural per gram of the gas phase;
the composition has a vaporization rate for conversion of the molecule in the condensed phase into the molecule in the gas phase;
the composition has a condensation rate for conversion of the molecule in the gas phase into the molecule in the condensed phase;
the composition has a mass transfer rate, which is equal to the vaporization rate minus the condensation rate; and
the mass transfer rate is a positive number.

7. The composition of claim 3, wherein:
the composition has a rate of sensible heat transfer from the gas phase to the condensed phase of at least 2 joules per gram of the condensed phase per second and no greater than 20 kilojoules per gram of the condensed phase per second;
the composition has a vaporization rate for conversion of the molecule in the condensed phase into the molecule in the gas phase;
the composition has a condensation rate for conversion of the molecule in the gas phase into the molecule in the condensed phase;
the composition has a mass transfer rate, which is equal to the vaporization rate minus the condensation rate; and
the mass transfer rate is a positive number.

8. The composition of claim 3, wherein:
the composition has a rate of sensible heat transfer from the gas phase to the condensed phase of at least 2 joules per gram of the condensed phase per second;
the composition has a vaporization rate for conversion of the molecule in the condensed phase into the molecule in the gas phase;
the composition has a condensation rate for conversion of the molecule in the gas phase into the molecule in the condensed phase;
the composition has a mass transfer rate, which is equal to the vaporization rate minus the condensation rate; and
the mass transfer rate is a positive number.

9. The composition of claim 3, wherein:
the composition has a rate of sensible heat transfer from the gas phase to the condensed phase of no greater than 20 kilojoules per gram of the condensed phase per second;
the composition has a vaporization rate for conversion of the molecule in the condensed phase into the molecule in the gas phase;
the composition has a condensation rate for conversion of the molecule in the gas phase into the molecule in the condensed phase;
the composition has a mass transfer rate, which is equal to the vaporization rate minus the condensation rate; and
the mass transfer rate is a positive number.

10. The composition of claim 3, wherein:
the condensed phase has a temperature that is less than the temperature of the gas phase; and
the composition has a rate of sensible heat transfer from the gas phase to the condensed phase of at least 2 joules per gram of the condensed phase per second.

11. The composition of claim 3, wherein:
the condensed phase has a temperature that is less than the temperature of the gas phase; and
the composition has a rate of sensible heat transfer from the gas phase to the condensed phase of no greater than 20 kilojoules per gram of the condensed phase per second.

12. The composition of claim 3, wherein:
the composition has a rate of sensible heat transfer from the gas phase to the condensed phase of at least 2 joules per gram of the condensed phase per second and no greater than 20 kilojoules per gram of the condensed phase per second;
the composition comprises no greater than 1 gram of the condensed phase per liter of the gas phase; and
the composition comprises no greater than 200 milligrams of the furfural per gram of the gas phase.

13. The composition of claim 3, wherein:
the composition has a rate of sensible heat transfer from the gas phase to the condensed phase of at least 2 joules per gram of the condensed phase per second;
the composition comprises no greater than 1 gram of the condensed phase per liter of the gas phase; and
the composition comprises no greater than 200 milligrams of the furfural per gram of the gas phase.

14. The composition of claim 3, wherein:
the composition has a rate of sensible heat transfer from the gas phase to the condensed phase of no greater than 20 kilojoules per gram of the condensed phase per second;
the composition comprises no greater than 1 gram of the condensed phase per liter of the gas phase; and
the composition comprises no greater than 200 milligrams of the furfural per gram of the gas phase.

15. The composition of claim 3, wherein the composition has a rate of sensible heat transfer from the gas phase to the condensed phase of at least 2 joules per gram of the condensed phase per second and no greater than 20 kilojoules per gram of the condensed phase per second.

16. The composition of claim 3, wherein the composition has a rate of sensible heat transfer from the gas phase to the condensed phase of at least 2 joules per gram of the condensed phase per second.

17. The composition of claim 3, wherein the composition has a rate of sensible heat transfer from the gas phase to the condensed phase of no greater than 20 kilojoules per gram of the condensed phase per second.

18. The composition of claim 3, wherein:
the composition has a vaporization rate for conversion of the molecule in the condensed phase into the molecule in the gas phase;
the composition has a condensation rate for conversion of the molecule in the gas phase into the molecule in the condensed phase;
the composition has a mass transfer rate, which is equal to the vaporization rate minus the condensation rate; and
the mass transfer rate is at least 5 micrograms of the molecule per gram of the condensed phase per second.

19. The composition of claim 3, wherein:
the composition has a vaporization rate for conversion of the molecule in the condensed phase into the molecule in the gas phase;
the composition has a condensation rate for conversion of the molecule in the gas phase into the molecule in the condensed phase;
the composition has a mass transfer rate, which is equal to the vaporization rate minus the condensation rate; and
the mass transfer rate is at least 5 micrograms of the molecule per gram of the composition per second.

20. The composition of claim 3, wherein:
the composition has a vaporization rate for conversion of the molecule in the condensed phase into the molecule in the gas phase;
the composition has a condensation rate for conversion of the molecule in the gas phase into the molecule in the condensed phase;
the composition has a mass transfer rate, which is equal to the vaporization rate minus the condensation rate; and
the mass transfer rate is a positive number.
